(19) United States
(12) Patent Application Publication
(10) Pub. No.: US 2023/0253106 A1
(43) Pub. Date: Aug. 10, 2023

(54) Electronic Stethoscope Interface Device

(71) Applicant: Iron Bow Technologies, LLC, Herndon, VA (US)

(72) Inventors: Anthony Mines, Sterling, VA (US); Peter Pistilli, Leesburg, VA (US); Hasso von Bredow, Fairfax, VA (US)

(21) Appl. No.: 17/666,000

(22) Filed: Feb. 7, 2022

(51) Int. Cl. G16H 40/67; A61B 7/04; A61B 5/00; A61B 7/02

(52) U.S. Cl. CPC G16H 40/67; A61B 7/04; A61B 5/0022; A61B 7/026

(57) ABSTRACT

An interface apparatus connects an electronic stethoscope to a computing device and an audio listening device. The apparatus has a housing that includes an audio input port to connect the stethoscope, an audio output port to connect the listening device, and a data transfer port to communicate with the computing device. An electronic circuit within the housing receives an audio signal from the stethoscope through the audio input port, outputs the audio signal to the listening device through the audio output port, and outputs the audio signal to the computing device through the data transfer port. An operating mode switch can toggle the interface apparatus between a first operating mode, where the listening device outputs sound from the stethoscope on one channel and sound from a remote location on a second channel, and a second operating mode, where the listening device outputs sound from the stethoscope on both channels.

### BACKGROUND

The present disclosure relates generally to telemedicine. In particular, the instant disclosure relates to an interface device allowing an electronic stethoscope to be used for telemedicine without requiring extensive configuration of supporting hardware to allow correct signal flow and operation

The term “telemedicine” refers to the remote consultation and diagnosis of patients by means of telecommunications technology. Telemedicine encompasses everything from a simple telephone call between two parties (e.g., doctor and patient) to a more sophisticated real-time videoconference between two parties (e.g., a medical professional, such as a nurse or nurse practitioner, located with a patient at a first, or “local,” site, and a physician located at a second, or “remote,” site).

Those of ordinary skill in the art will be familiar with various telemedicine platforms. These platforms include, without limitation, the CLINiC™, MedView™, and eCLINiC™ clinical care devices and the MEDiC™ mobile examination device, all from Iron Bow Technologies (Herndon, Va.). These specialized systems are typically used in commercial health care settings and are purpose-built for telemedicine applications.

The use of telemedicine increased considerably during the coronavirus (COVID-19) pandemic that began in early 2020. Notably, with the capacity of the health care system focused on treating COVID-19 patients and many individuals reluctant to seek in-person care for fear of contracting COVID-19, telemedicine began to shift from use of purpose-built telemedicine hardware and software to more widely-available hardware and software. This paradigm shift includes the use of consumer electronics, such as personal computers and tablets, and free-to-use audio- and video-conferencing software, such as Zoom or Microsoft Teams, for telemedicine purposes. Even when the COVID-19 pandemic abates, these trends are expected to continue.

There are, nevertheless, technical challenges associated with using both consumer and commercial hardware and software applications, which were primarily designed for face-to-face conversations and business meetings, for telemedicine purposes. Many of these challenges relate to the inability to correctly use specialized examination and/or diagnostic devices with systems running general-purpose audio- and video-conferencing software.

For instance, an electronic stethoscope typically outputs standard line level audio signal, which must be simultaneously transmitted to the remote site and heard by the stethoscope user at the local site. Further complications arise because the remote site listener needs to hear the output of the stethoscope without hearing any disturbing sounds, such as a patient talking, from the local site. It is also necessary for the remote site listener to be able to instruct the local site stethoscope user, for example, to direct them where to place the stethoscope on the patient's body, without such instructions being heard by the patient.

Most personal computers and tablets, as well as many commercial bespoke videoconferencing systems, however, include neither a suitable line audio input nor routing facilities and audio output to feed the remote site physician's instructions to the local site user. These pre-requisite audio input/output and routing requirements may make it impossible to connect the electronic stethoscope to a personal computer or tablet for use during a telemedicine visit.

Complications exist even with respect to devices that include the requisite audio input/output. Indeed, it is not enough for a user simply to connect the electronic stethoscope to his or her device of choice. Instead, once connected, the user must also configure not only the operating system, but also the audio- and video-conferencing software to be used, before the electronic stethoscope can be used for a telemedicine visit.

Put another way, there is no extant “plug-and-play” solution to interface examination and/or diagnostic devices useful for telemedicine with the type of computing hardware and software that is becoming increasingly prevalent in telemedicine.

### BRIEF SUMMARY

Disclosed herein is an apparatus for interfacing an electronic stethoscope to a computing device and an audio listening device. The apparatus includes a housing including a plurality of communication ports, the plurality of communication ports in turn including: an audio input port configured for communication with the electronic stethoscope; an audio output port configured for communication with the audio listening device; and a data transfer port configured for communication with the computing device. The apparatus also includes an electronic circuit disposed within the housing and in operable communication with the plurality of communication ports. The electronic circuit is configured to: receive an audio signal from the electronic stethoscope through the audio input port; output the audio signal to the audio listening device through the audio output port; and output the audio signal to the computing device through the data transfer port.

In certain embodiments, the apparatus further includes an operating mode switch operable to toggle the apparatus between a first operating mode and a second operating mode. When the apparatus is in the first operating mode, the electronic circuit is configured to: output the audio signal to the audio listening device through the audio output port on a first audio channel; receive a secondary audio signal from the computing device through the data transfer port; and output the secondary audio signal to the audio listening device through the audio output port on a second audio channel. When the apparatus is in the second operating mode, the electronic circuit is configured to output the audio signal to the audio listening device through the audio output port on the first audio channel and the second audio channel.

The electronic circuit can include a digital-to-analog converter between the data transfer port and the audio output port, and the secondary audio signal can pass through the digital-to-analog converter prior to being output to the audio listening device through the audio output port on the second audio channel. Similarly, the electronic circuit can include an analog-to-digital converter between the audio input port and the data transfer port, and the audio signal can pass through the analog-to-digital converter prior to being output to the computing device through the data transfer port.

It is also contemplated that the apparatus include a power state switch operable to toggle the apparatus between a power-off state and a power-on state.

The data transfer port can include a universal serial bus (USB) connection and/or a wireless connection. Similarly, at least one of the audio input port and the audio output port can include a wireless connection.

Also disclosed herein is a method of interfacing an electronic stethoscope to a computing device through an interface apparatus including an audio input port for communication with the electronic stethoscope, an audio output port for communication with an audio listening device, and a data transfer port for communication with the computing device. The method includes: the interface apparatus receiving an audio signal from the electronic stethoscope through the audio input port; the interface apparatus outputting the audio signal to the audio listening device through the audio output port; and the interface apparatus outputting the audio signal to the computing device through the data transfer port.

The step of the interface apparatus receiving the audio signal from the electronic stethoscope through the audio input port can include the interface apparatus receiving an analog audio signal from the electronic stethoscope through the audio input port. In turn, the step of the interface apparatus outputting the audio signal to the computing device through the data transfer port can include: the interface apparatus converting the analog audio signal a digital audio signal; and the interface apparatus outputting the digital audio signal to the computing device through the data transfer port.

The step of the interface apparatus outputting the audio signal to the audio listening device through the audio output port can include the interface apparatus outputting the audio signal to the audio listening device through the audio output port on a first channel. The method can further include: the interface apparatus receiving a secondary audio signal from the computing device through the data transfer port; and the interface apparatus outputting the secondary audio signal to the audio listening device through the audio output port on a second channel.

Further, the step of the interface apparatus receiving the secondary audio signal from the computing device through the data transfer port can include the interface apparatus receiving a digital secondary audio signal from the computing device through the data transfer port. In turn, the step of the interface apparatus outputting the secondary audio signal to the audio listening device through the audio output port on the second channel can include: the interface apparatus receiving converting the digital secondary audio signal from an analog secondary audio signal; and the interface apparatus outputting the analog secondary audio signal to the audio listening device through the audio output port.

In certain aspects of the disclosure, the method further includes the interface apparatus suppressing audible outputs from and/or audible inputs to the computing device.

The instant disclosure also provides a telemedicine system, including: a computing device configured for audio-visual communication with a remote location; an electronic stethoscope; an audio listening device; and an interface apparatus interconnecting the electronic stethoscope, the audio listening device, and the computing device. The interface apparatus includes: a housing including an audio input port, an audio output port, and a bi-directional data transfer port; and an electronic circuit disposed within the housing; wherein an output of the electronic stethoscope is coupled to the audio input port, wherein an input of the audio listening device is coupled to the audio output port, wherein the computing device is coupled to the interface apparatus through the bi-directional data transfer port. The electronic circuit is configured to: receive an audio signal from the electronic stethoscope; output the audio signal to the audio listening device; and output the audio signal to the computing device for transmission by the computing device to the remote location.

The interface apparatus can include an operating mode switch operable to toggle the interface apparatus between a first operating mode and a second operating mode. When the interface apparatus is in the first operating mode, the electronic circuit can be configured to: output the audio signal to the audio listening device on a first audio channel; receive a secondary audio signal, transmitted to the computing device from the remote site, from the computing device; and output the secondary audio signal to the audio listening device on a second audio channel. When the interface apparatus is in the second operating mode, the electronic circuit can be configured to output the audio signal to the audio listening device on the first audio channel and the second audio channel.

The electronic circuit can include at least one of: an analog-to-digital converter configured to convert the audio signal from an analog audio signal to a digital audio signal prior to outputting the audio signal to the computing device for transmission to the remote location; and a digital-to-analog converter configured to convert the secondary audio signal from a digital audio signal to an analog audio signal prior to outputting the secondary audio signal to the audio listening device on the second channel.

The electronic circuit can further be configured to suppress audible outputs from the computing device and/or audible inputs to the computing device.

The bi-directional data transfer port can include a universal serial bus (USB) connection and/or a wireless connection.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a block diagram of a telemedicine system according to aspects of the instant disclosure.

FIG. 2 depicts an embodiment of an interface apparatus as disclosed herein.

FIG. 3 is a functional block circuit diagram of the interface apparatus shown in FIG. 2.

While multiple embodiments are disclosed, still other embodiments of the present disclosure will become apparent to those skilled in the art from the following detailed description, which shows and describes illustrative embodiments. Accordingly, the drawings and detailed description are to be regarded as illustrative in nature and not restrictive.

### DETAILED DESCRIPTION

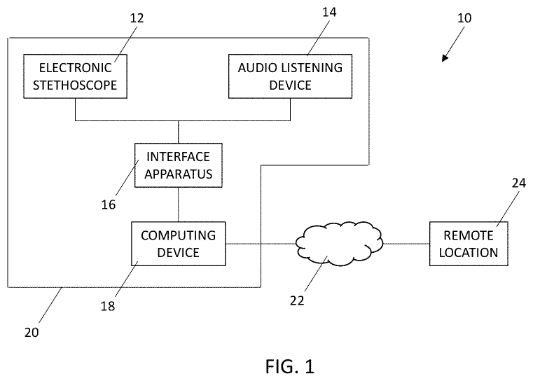

FIG. 1 is a block diagram of a telemedicine system 10 according to aspects of the instant disclosure. As shown in FIG. 1, telemedicine system 10 generally includes an electronic stethoscope 12, an audio listening device 14, an interface apparatus 16, and a computing device 18, all located at a first location 20, such as a clinic or other healthcare facility. First location 20, and, in particular, computing device 18, is connected over a network 22, such as the Internet, to a remote location 24, such as a physician's office.

Computing device 18 enables bi-directional audio-visual communication between individuals at first location 20 (e.g., a patient and a medical practitioner) and individuals at remote location 24 (e.g., a remote medical practitioner using a counterpart computing device at remote location 24).

Advantageously, interface apparatus 16 according to the instant teachings enables and enhances the use of a wide range of other computing devices 18, including videoconferencing systems (e.g., the Poly Studio X30 video bar from Plantronics, Inc. (Santa Cruz, Calif.) or the Webex® Room Kit Mini video conference system from Cisco Systems (San Jose, Calif.)), smartphones, tablet computers, laptop computers, desktop computers, and the like, for telemedicine. Indeed, the following description will make clear to the ordinarily-skilled artisan that the scope of computing device 18 encompasses any hardware and/or software capable of bi-directional audio-visual communication with remote location 24 and possessing a suitable data transfer port for bi-directional communication with interface apparatus 16. This greatly enhances the practicality and availability of telemedicine by increasing the range of devices usable for telemedicine visits.

Audio listening device 14 can be any device capable of outputting audio. According to aspects of the instant disclosure, audio listening device 14 includes over-ear headphones, which desirably provide a listener with increased isolation from ambient noise, thus improving the listener's ability to hear sound from electronic stethoscope 12 and/or instructions from an individual at remote location 24. It should be understood, however, that other devices (e.g., on-ear headphones, in-ear headphones, earbuds, or speakers) can also be used as audio listening device 14 without departing from the scope of the instant disclosure.

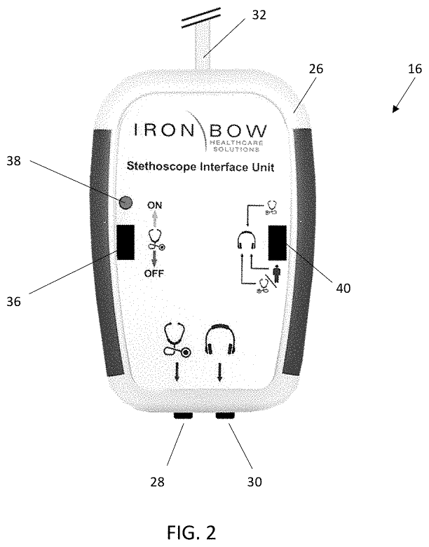

Interface apparatus 16 interconnects electronic stethoscope 12, audio listening device 14, and computing device 18. As shown to good advantage in FIG. 2, interface apparatus 16 includes a housing 26 that incorporates a plurality of communication ports, namely, an audio input port 28, an audio output port 30, and a data transfer port 32. Electronic stethoscope 12 can be connected to audio input port 28, while audio listening device 14 can be connected to audio output port 30. Although audio input port 28 and audio output port 30 are both shown as wired connections (e.g., standard 3.5 mm line audio connections), either or both could also be implemented wirelessly (e.g., as a Bluetooth audio connection) without departing from the scope of the present teachings.

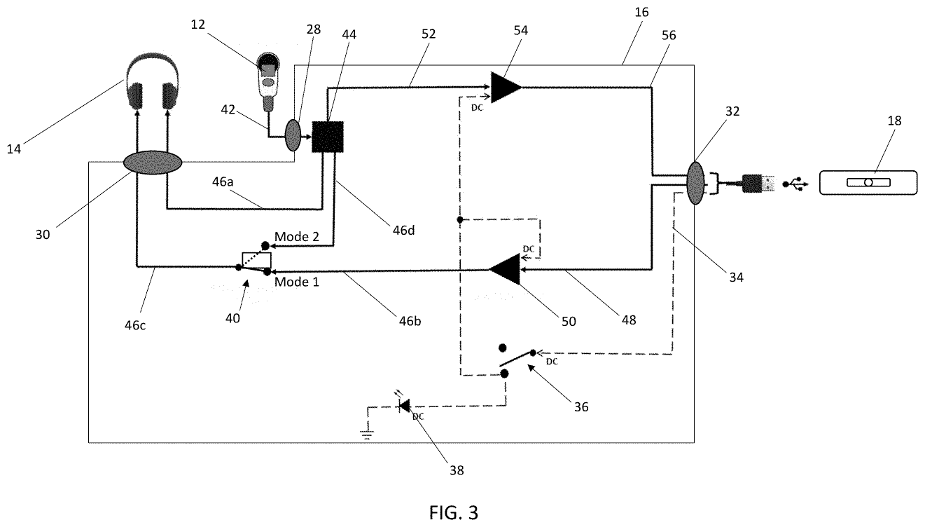

Bi-directional communication between interface apparatus 16 and computing device 18 is enabled over data transfer port 32. In certain embodiments, data transfer port 32 is a universal serial bus (USB) connection. Advantageously, the use of a USB connection allows computing device 18 to supply DC power to interface apparatus 16 in accordance with the applicable specification(s). A USB power connection is shown in dashed lines 34 in FIG. 3. It should be understood, however, that data transfer port 32 could alternatively enable bi-directional wireless communication between interface apparatus 16 and computing device 18, though this would require an alternative power source, such as one or more batteries, for interface apparatus 16.

Interface apparatus 16 includes a power state switch 36. Power state switch 36 is operable to toggle interface apparatus 16 between a power-on state and a power-off state. When interface apparatus 16 is in the power-on state, an indicator 38, such as an LED, can illuminate.

Interface apparatus further includes an operating mode switch 40. As discussed in greater detail below, operating mode switch 40 allows a user to toggle interface apparatus 16 between a first operating mode, in which the user hears audio from electronic stethoscope 12 on one channel of audio listening device 14 and audio from remote location 24 on another channel of audio listening device 14, and a second operating mode, in which the user hears audio from electronic stethoscope 12 on both channels of audio listening device 14.

Electronic circuitry to enable operation of interface apparatus 16 is disposed within housing 26. Operation of interface apparatus 16 will be explained with reference to the functional block diagram of this electronic circuitry in FIG. 3.

As illustrated in FIG. 3, electronic stethoscope is connected to audio input port 28, audio listening device is connected to audio output port 30, and interface apparatus 16 is connected to computing device 18 (e.g., via a USB port on computing device 18). Interface apparatus 16 is then placed in the power-on state using power state switch 36; indicator 38 illuminates accordingly. The user can also select between the two operating modes described above using operating mode switch 40.

Assume first that the user places operating mode switch 40 in the “Mode 1” operating position, as shown by a solid line in FIG. 3. In this operating mode, interface apparatus 16 receives an audio signal from electronic stethoscope 12, through audio input port 28, on line 42.

Typically, the audio signal output by electronic stethoscope 14 and received as input by interface apparatus 16 on line 42 is an analog line-level signal. A distribution device 44 outputs the audio signal (e.g., as an analog line-out signal) through audio output port 30 on line 46a, feeding one channel of audio listening device 14 (e.g., within the right earcup of over-ear headphones).

Interface apparatus 16 further receives an audio signal, referred to herein as a “secondary audio signal” to distinguish it from the audio signal received from electronic stethoscope 12, from computing device 18, through data transfer port 32, on line 48. For example, the secondary audio signal received on line 48 can be an audio communication from a physician at remote location 24, instructing the user at first location 20 to reposition electronic stethoscope 12 on the patient's chest.

Typically, the audio signal output by computing device 18 and received as input by interface apparatus 16 on line 48 is a digital audio signal. Thus, a digital-to-analog converter 50 can convert the signal to an analog audio signal prior to outputting it (e.g., as an analog line-out signal) through audio output port 30 on lines 46b (before operating mode switch 40) and 46c (after operating mode switch 40), feeding a second channel of audio listening device 14 (e.g., within the left earcup of over-ear headphones).

Thus, in “Mode 1,” a user at first location 20 will hear audio from electronic stethoscope 12 in one ear and audio from computing device 18 (e.g., from the telemedicine session with remote location 24) in the other ear.

Assume now that the user places operating mode switch 40 in the “Mode 2” operating position, as shown by a dashed line in FIG. 3. In this mode, distribution device 44 passes the audio signal received from electronic stethoscope 12 through both line 46a and lines 46d (before operating mode switch 40) and 46c (after operating mode switch 40) for output on both channels of audio listening device 14. Thus, in “Mode 2,” a user at first location 20 will hear audio from electronic stethoscope 12 in both ears.

In both operating modes, distribution device 44 also passes the audio signal from electronic stethoscope 12 along line 52 to an analog-to-digital converter 54, which outputs a digital signal for communication to computing device 18, through data transfer port 32, on line 56. Computing device 18 can, in turn, send the now-digitized audio signal from electronic stethoscope 12 over network 22 to remote location 24.

In addition, when interface device 16 is attached to computing device 18 and in the power-on state, other audible inputs to and outputs from computing device 18 will be disabled (or “suppressed”). Thus, for example, any microphones and/or speakers built into or otherwise connected to computing device 18 will not operate with interface device 16 connected and powered on. Disabling or suppressing other audible outputs ensures that only the user of audio listening device 14 can hear the sound from electronic stethoscope 12 and, in “Mode 1,” from remote location 24. Likewise, disabling or suppressing other audible inputs ensures ambient noise is not captured.

Most advantageously, this suppression happens automatically when computing device 18 detects (e.g., in accordance with the applicable USB specification(s)) that interface device 16 has been connected and powered on. The user at first location 20 is not required to reconfigure any settings within the operating system of computing device 18 to select and/or disable certain audio inputs and/or outputs.

Conversely, when interface device 16 is placed into the power-off state, or otherwise disconnected from computing device 18, the previous audio state of computing device 18 can be restored automatically. That is, any previously-selected microphone and speaker attached or incorporated into computing device 18 will be re-activated.

Although several embodiments have been described above with a certain degree of particularity, those skilled in the art could make numerous alterations to the disclosed embodiments without departing from the spirit or scope of this invention.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. An apparatus for interfacing an electronic stethoscope to a computing device and an audio listening device, the apparatus comprising:
a housing including a plurality of communication ports, the plurality of communication ports comprising:
an audio input port configured for communication with the electronic stethoscope;
an audio output port configured for communication with the audio listening device; and
a data transfer port configured for communication with the computing device; and
an electronic circuit disposed within the housing and in operable communication with the plurality of communication ports, the electronic circuit configured to:
receive an audio signal from the electronic stethoscope through the audio input port;
output the audio signal to the audio listening device through the audio output port; and
output the audio signal to the computing device through the data transfer port.

2. The apparatus according to claim 1, further comprising an operating mode switch operable to toggle the apparatus between a first operating mode and a second operating mode, wherein, when the apparatus is in the first operating mode, the electronic circuit is configured to:
output the audio signal to the audio listening device through the audio output port on a first audio channel;
receive a secondary audio signal from the computing device through the data transfer port; and
output the secondary audio signal to the audio listening device through the audio output port on a second audio channel; and
wherein, when the apparatus is in the second operating mode, the electronic circuit is configured to output the audio signal to the audio listening device through the audio output port on the first audio channel and the second audio channel.

3. The apparatus according to claim 2, wherein the electronic circuit comprises a digital-to-analog converter between the data transfer port and the audio output port, and wherein the secondary audio signal passes through the digital-to-analog converter prior to being output to the audio listening device through the audio output port on the second audio channel.

4. The apparatus according to claim 1, further comprising a power state switch operable to toggle the apparatus between a power-off state and a power-on state.

5. The apparatus according to claim 1, wherein the electronic circuit comprises an analog-to-digital converter between the audio input port and the data transfer port, and wherein the audio signal passes through the analog-to-digital converter prior to being output to the computing device through the data transfer port.

6. The apparatus according to claim 1, wherein the data transfer port comprises a universal serial bus (USB) connection.

7. The apparatus according to claim 1, wherein the data transfer port comprises a wireless connection.

8. The apparatus according to claim 1, wherein at least one of the audio input port and the audio output port comprises a wireless connection.

9. A method of interfacing an electronic stethoscope to a computing device through an interface apparatus including an audio input port for communication with the electronic stethoscope, an audio output port for communication with an audio listening device, and a data transfer port for communication with the computing device, the method comprising:
the interface apparatus receiving an audio signal from the electronic stethoscope through the audio input port;
the interface apparatus outputting the audio signal to the audio listening device through the audio output port; and
the interface apparatus outputting the audio signal to the computing device through the data transfer port.

10. The method according to claim 9, wherein:
the interface apparatus receiving the audio signal from the electronic stethoscope through the audio input port comprises the interface apparatus receiving an analog audio signal from the electronic stethoscope through the audio input port; and
the interface apparatus outputting the audio signal to the computing device through the data transfer port comprises:
the interface apparatus converting the analog audio signal a digital audio signal; and
the interface apparatus outputting the digital audio signal to the computing device through the data transfer port.

11. The method according to claim 9, wherein the interface apparatus outputting the audio signal to the audio listening device through the audio output port comprises the interface apparatus outputting the audio signal to the audio listening device through the audio output port on a first channel, the method further comprising:
the interface apparatus receiving a secondary audio signal from the computing device through the data transfer port; and
the interface apparatus outputting the secondary audio signal to the audio listening device through the audio output port on a second channel.

12. The method according to claim 11, wherein:
the interface apparatus receiving the secondary audio signal from the computing device through the data transfer port comprises the interface apparatus receiving a digital secondary audio signal from the computing device through the data transfer port; and
the interface apparatus outputting the secondary audio signal to the audio listening device through the audio output port on the second channel comprises:
the interface apparatus receiving converting the digital secondary audio signal from an analog secondary audio signal; and
the interface apparatus outputting the analog secondary audio signal to the audio listening device through the audio output port.

13. The method according to claim 9, further comprising the interface apparatus suppressing audible outputs from the computing device.

14. The method according to claim 9, further comprising the interface apparatus suppressing audible inputs to the computing device.

15. A telemedicine system, comprising:
a computing device configured for audio-visual communication with a remote location;
an electronic stethoscope;
an audio listening device; and
an interface apparatus interconnecting the electronic stethoscope, the audio listening device, and the computing device, wherein the interface apparatus comprises:
a housing including an audio input port, an audio output port, and a bi-directional data transfer port; and
an electronic circuit disposed within the housing;
wherein an output of the electronic stethoscope is coupled to the audio input port,
wherein an input of the audio listening device is coupled to the audio output port,
wherein the computing device is coupled to the interface apparatus through the bi-directional data transfer port, and
wherein the electronic circuit is configured to:
receive an audio signal from the electronic stethoscope;
output the audio signal to the audio listening device; and
output the audio signal to the computing device for transmission by the computing device to the remote location.

16. The telemedicine system according to claim 15:
wherein the interface apparatus further comprises an operating mode switch operable to toggle the interface apparatus between a first operating mode and a second operating mode,
wherein, when the interface apparatus is in the first operating mode, the electronic circuit is configured to:
output the audio signal to the audio listening device on a first audio channel;
receive a secondary audio signal, transmitted to the computing device from the remote site, from the computing device; and
output the secondary audio signal to the audio listening device on a second audio channel; and
wherein, when the interface apparatus is in the second operating mode, the electronic circuit is configured to output the audio signal to the audio listening device on the first audio channel and the second audio channel.

17. The telemedicine system according to claim 16, wherein the electronic circuit comprises at least one of:
an analog-to-digital converter configured to convert the audio signal from an analog audio signal to a digital audio signal prior to outputting the audio signal to the computing device for transmission to the remote location; and
a digital-to-analog converter configured to convert the secondary audio signal from a digital audio signal to an analog audio signal prior to outputting the secondary audio signal to the audio listening device on the second channel.

18. The telemedicine system according to claim 15, wherein the electronic circuit is further configured to suppress audible outputs from the computing device and audible inputs to the computing device.

19. The telemedicine system according to claim 15, wherein the bi-directional data transfer port comprises a universal serial bus (USB) connection.

20. The telemedicine system according to claim 15, wherein at least one of the audio input port and the audio output port comprises a wireless connection.
